(19) United States
(12) Patent Application Publication
(10) Pub. No.: US 2024/0116883 A1
(43) Pub. Date: Apr. 11, 2024

(54) CANNABINOID DERIVATIVES AND THEIR USE THE TREATMENT OF INFLAMMATION AND/OR PAIN AND/OR OBESITY

(71) Applicant: Yissum Research Development Company of The Hebrew University of Jerusalem Ltd., Jerusalem (IL)

(72) Inventors: Ruth GALLILY, Jerusalem (IL); Raphael MECHOULAM, Jerusalem (IL); Aviva BREUER, Jerusalem (IL); Natalya M KOGAN, Kfar Adumim (IL); Yarden LAVI, Rishon Lezion (IL); Zhanna YEKHTIN, Jerusalem (IL)

(73) Assignee: Yissum Research Development Company of The Hebrew University of Jerusalem Ltd., Jerusalem (IL)

(21) Appl. No.: 18/038,317

(22) PCT Filed: Nov. 24, 2021
(86) PCT No.: PCT/IL2021/051398

§ 371 Date: May 23, 2023

Related U.S. Application Data
(60) Provisional application No. 63/117,465, filed on Nov. 24, 2020.

(51) Int. Cl. C07D 265/30; A61P 29/00; C07C 39/17; C07C 39/23; C07C 43/23

(52) U.S. Cl. CPC C07D 265/30; A61P 29/00; C07C 39/17; C07C 39/23; C07C 43/23; C07C 2601/16

(57) ABSTRACT

The invention provides cannabinoid derivatives, methods of their preparation, compositions comprising them and uses thereof in the treatment of inflammation and/or pain and/or obesity.

### BACKGROUND OF THE INVENTION

Cannabidiol (CBD) is the major non-psychotropic cannabinoid in most cannabis preparations, such as hashish and marihuana. However, CBD causes none of the psychotropic effects typical of Δ9-tetrahydrocannabinol (Δ9-THC) since CBD does not bind to the known cannabinoid receptors CB1 or CB2, and therefore does not cause the central or peripheral effects mediated by these receptors. Nonetheless, CBD has been shown in in-vitro assays, as well as in in-vivo assays in animals and in some human, preliminary trials, to produce numerous pharmacological effects, some of which are of high potential therapeutic value.

Thus, for example, CBD has been found to produce several, potentially therapeutic, effects in animal models, as well as in patients with neurological diseases, in anxiety and psychosis. CBD was also found to be a neuroprotective antioxidant.

Some reports describe the in-vitro effects of CBD on immune cells, such as the inhibition of nitric oxide (NO) production by mouse peritoneal macrophages and the suppression of TNF-α, IL-1α and IFNγ by human peripheral blood mononuclear cells.

These in-vitro studies lend support to earlier reports on analgesic and anti-inflammatory effects of CBD in animals. CBD was found much more potent than aspirin in the phenylbenzoquinone writhing test in mice in a standard analgesic activity assay. In the tetradecanoylphorbolacetate (TPA) induced erythema of mouse ear (a standard anti-inflammatory activity assay) CBD caused 92% inhibition of the inflammation response on application of a 100 pg/ml solution.

Some studies provided CBD, resorcinol and other cannabinoid derivatives with a wide scope of indications [1-24]. U.S. Pat. No. 3,661,919 provided resorcinol derivatives exhibiting bactericidal and fungicidal activities. U.S. Pat. No. 4,018,777 provided resorcinol derivatives useful as tranquilizers, analgesics, sedative-hypnotics and anticonvulsants. U.S. Pat. No. 6,274,635 provided 5-alkyl- resorcinol derivatives, cannabinol derivatives, cannabidiol derivatives and cannabigerol derivatives for treating diseases of the immune system. U.S. Patent Application No. 20020137802 provided cannabinoid or resorcinolic derivatives for inhibiting the activation and/or aggregation of blood platelets. U.S. Pat. No. 6,566,560 provided resorcinol derivatives to attenuate the growth of a neoplasm. U.S. Patent Application No. 20030232101 provided cannabinol derivatives, Δ9-THC derivatives, cannabichromene derivatives, cannabidiol derivatives and cannabigerol derivatives for preventing the transmission of HIV. U.S. Pat. No. 7,105,685 and U.S. Patent Application No. 20070179135 provided cannabinol derivatives and pharmaceutical preparations thereof. U.S. Patent Application No. 20040034108, WO 2004016246 and British Patent No. GB2392093 provided cannabinoid derivatives for administration via a pump action spray. U.S. Pat. Nos. 7,179,800 and 7,285,687 and WO 2003091189 provided cannabidiol derivatives useful in the treatment of pain, inflammation and autoimmune disease. U.S. Pat. No. 6,630,507 and WO 199953917 provided cannabinoid derivatives as antioxidants and neuroprotectants.

U.S. Pat. Nos. 4,282,248 and 6,410,588, U.S. Patent Application Nos. 20030166727 and 20070082954, as well as WO 2005023741 and WO 2001095899, which are incorporated in their entirety as if fully set forth herein, teach cannabidiol derivatives, some of which have been synthesized and biologically evaluated [25, 21], and some of which have been shown to possess anti-inflammatory activity.

### SUMMARY OF THE INVENTION

The present invention provides a compound of general formula (I), including any stereoisomer, solvate or salt thereof:

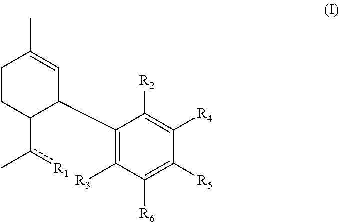

Wherein [symbol] is a single or double bond; R1 is selected from CH3 and CH2; R2 and R3 are selected from OH, O(C1-C3 alkyl), straight or branched C3-C10 alkyl; R4 is a straight or branched C1-C5 alkyl; R5 is selected from OH, straight or branched C3-C10 alkyl; R6 is selected from H, straight or branched C1-C5 alkyl; Provided that when [symbol] is a double bond at least one of R2 and R3 is different than OH; or R6 is different than H; and provided that when R3 and R5 are both OH than R2 is C5 alkyl and R4 is C1-C5 alkyl.

In some embodiments [symbol] is a single bond.

In some embodiments [symbol] is a single bond; R2 and R3 are both OH.

In some embodiments R2 and R3 are each O(C1-C3 alkyl).

In some embodiments R2 is a straight or branched C3-C10 alkyl.

In some embodiments R3 and R5 are both OH.

In some embodiments R3 and R5 are both OH; R2 is a straight or branched C3-C10 alkyl; R4 is straight or branched C1-C5 alkyl.

In some embodiments R4 and R6 are each C1-C5 alkyl.

In some embodiments a compound of formula (I), is selected from: (1′S,2′S)-2′-isopropyl-3,5′-dimethyl-4-pentyl-1′,2′,3′,4′-tetrahydro-[1,1′-biphenyl]-2,6-diol (HUM-217);

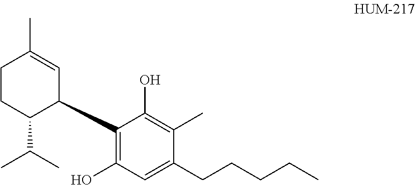 [SMILES: CCCCCc1cc(O)c([C@@H]2C=C(C)CC[C@H]2C(C)C)c(O)c1C]

(1R,2R)-2′,6′-dimethoxy-3′,5-dimethyl-4′-pentyl-2-(prop-1-en-2-yl)-1,2,3,4-tetrahydro-1,1′-biphenyl (HUM-219);

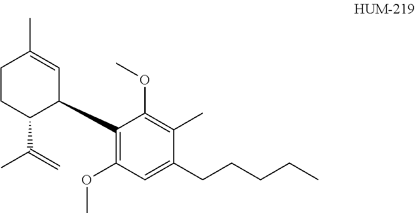 [SMILES: C=C(C)[C@@H]1CCC(C)=C[C@H]1c1c(OC)cc(CCCCC)c(C)c1OC]

(1′R,2′R)-5,5′-dimethyl-6-pentyl-2′-(prop-1-en-2-yl)-1′,2′,3′,4′-tetrahydro-[1,1′-biphenyl]-2,4-diol (HUM-229);

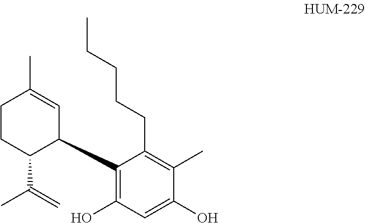 [SMILES: C=C(C)[C@@H]1CCC(C)=C[C@H]1c1c(O)cc(O)c(C)c1CCCCC]

(1′R,2′R)-3,5,5′-trimethyl-4-pentyl-2′-(prop-1-en-2-yl)-1′,2′,3′,4′-tetrahydro-[1,1′-biphenyl]-2,6-diol (HUM-236)

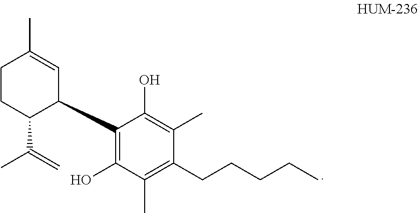 [SMILES: C=C(C)[C@@H]1CCC(C)=C[C@H]1c1c(O)c(C)c(CCCCC)c(C)c1O]

In some embodiments a compound of general formula (I) is compound of general formula (I′) wherein substituents R1-R6 are defined as above:

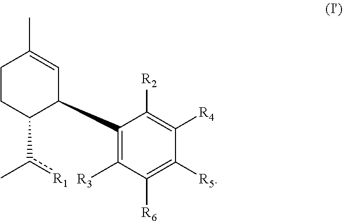

The invention further provides a compound of general formula (V), including any stereoisomer, diastereomer, solvate, or salt thereof:

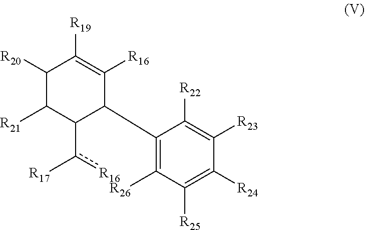

Wherein [symbol] is a single or double bond; R16 is selected from straight or branched C1-C5 alkyl; straight or branched C2-C8 alkenyl; R17-R21 are each selected from H, straight or branched C1-C5 alkyl; straight or branched C2-C8 alkenyl; straight or branched C2-C8 alkynyl; amine, halo, haloalkyl, OH, alkoxy, aryloxy, thiohydroxy, thioalkoxy, thioaryloxy, carboxy, amide, thioamide, thioimide, CN; R22 and R26 are each selected from OH, O(C1-C3 alkyl; R23, R24 and R25 are each selected from a straight or branched C1-C5 alkyl; H; Provided that when [symbol] is a double bond at least one of R22 and R26 is different than OH; and R25 is different than H; and provided that at least one of R18-R21 is different than H; and provided that when R24 and R26 are both OH than R22 is C5 alkyl and R23 is C1-C5 alkyl.

In some embodiments a compound of formula (V) is a compound of general formula (V′):

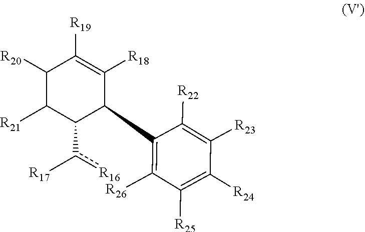

Wherein each of R16-R26 are defined as above.

The invention further provides a compound of general formula (II), including any stereoisomer, solvate or salt thereof:

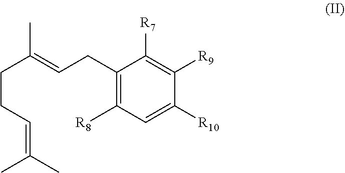

Wherein R7 and R8 are each selected from OH, O(C1-C3 alkyl), OC(═O)R16; R9 is selected from H, or C1-C3 alkyl; R10 is a straight or branched C3-C10 alkyl; R16 is C1-C5 alkyl optionally substituted by at least one of morpholino (O(CH2CH2)2 NH) derivative; Provided that when both R7 and R8 are OH, then R9 is different than C1 alkyl.

In some embodiments at least one of R7 and R8 is O(C1-C3 alkyl).

In some embodiments at least one of R7 and R8 is OC(═O)R16 wherein R16 is C1-C5 alkyl optionally substituted by at least one of morpholino (O(CH2CH2)2 NH) derivative.

In some embodiments both of R7 and R8 are O(C1-C3 alkyl).

In some embodiments a compound of general formula (II) is selected from: (E)-2-(3,7-dimethylocta-2,6-dien-1-yl)-3-methoxy-5-(2-methyloctan-2-yl)phenol (HUM-223),

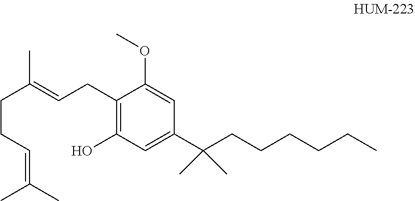

(E)-2-(3,7-dimethylocta-2,6-dien-1-yl)-3-methoxy-5-pentylphenyl 3-morpholino propanoate (HUM-233),

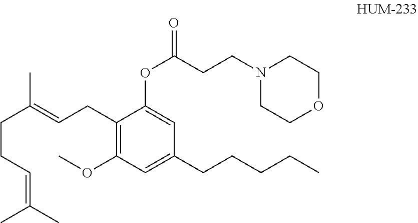 [SMILES: CCCCCc1cc(OC)c(C/C=C(\C)CCC=C(C)C)c(OC(=O)CCN2CCOCC2)c1]

(E)-4-(3-(2-(3,7-dimethylocta-2,6-dien-1-yl)-3-methoxy-5-pentylphenoxy)-3-oxopropyl) morpholin-4-ium (Z)-3-carboxyacrylate (HUM-234),

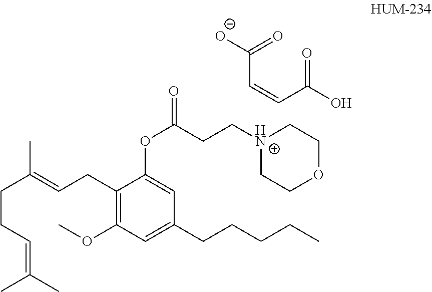

(E)-2-(3,7-dimethylocta-2,6-dien-1-yl)-1,3-dimethoxy-4-methyl-5-pentylbenzene (HUM-235)

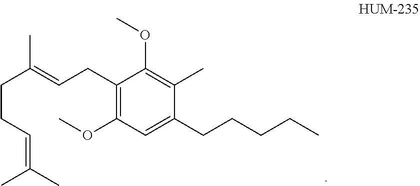 [SMILES: CCCCCc1cc(OC)c(C/C=C(\C)CCC=C(C)C)c(OC)c1C]

The invention further provides a compound of general formula (III), including a salt, solvate or stereoisomer thereof:

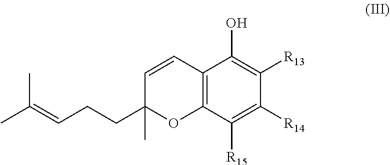

Wherein R13, R14 and R15 are each selected from H, straight or branched C1-C10 alkyl; provided that when R13 is CH3, R15 is different than H.

In some embodiments R13 is CH3.

In some embodiments R13 is H.

In some embodiments R15 is CH3.

In some embodiments R15 is H.

In some embodiments R14 is a straight or branched C3-C10 alkyl.

In some embodiments, a compound of general formula (III) is 2,8-dimethyl-2-(4-methylpent-3-en-1-yl)-7-pentyl-2H-chromen-5-ol (HUM-238)

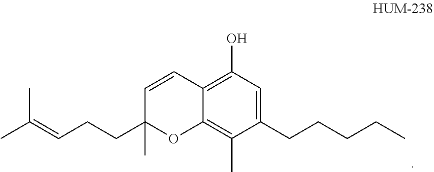

The invention further provides a compound of general formula (IV), including any stereoisomer, solvate or salt thereof:

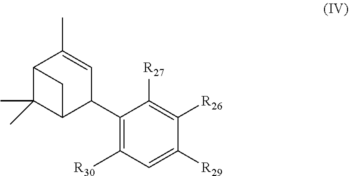

Where R27 and R30 are each selected from OH, O(C1-C3 alkyl); R28 and R29 are each independently a straight or branched C1-C10 alkyl.

In some embodiments, at least one of R27 and R30 is OH. In other embodiments, R27and R30 are both OH.

In some embodiments, a compound of general formula (IV), including any stereoisomer, solvate or salt thereof is a compound of general formula (IV′):

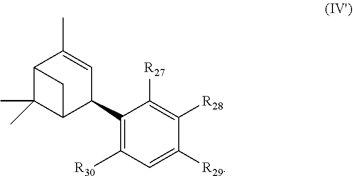

In some embodiments, a compound of general formula (IV) is selected from: 4-methyl-5-pentyl-2-((2R)-4,6,6-trimethylbicyclo[3.1.1]hept-3-en-2-yl)benzene-1,3-diol (HUM-231)

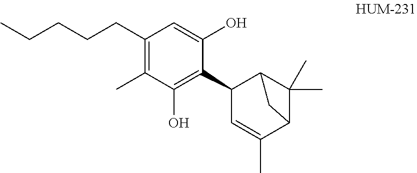

The invention further provides a compound of general formula (IV), including any stereoisomer, solvate or salt thereof, wherein R27 and R30 are each selected from OH, O(C1-C3 alkyl), C1-C10 alkyl; R28 and R29 are each independently a straight or branched OH, C1-C10 alkyl.

In some embodiments a compound of the present invention is 4-methyl-5-pentyl-6-((2R)-4,6,6-trimethylbicyclo[3.1.1]hept-3-en-2-yl)benzene-1,3-diol (HUM-332)

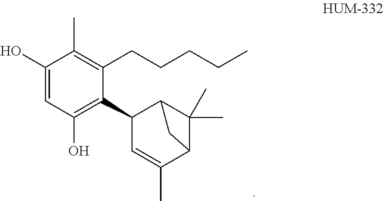 [SMILES: CCCCCc1c(C)c(O)cc(O)c1[C@@H]1C=C(C)C2CC1C2(C)C]

The invention provides a pharmaceutical composition comprising at least one compound as disclosed herein above and below of general formula (I), (II), (III) and (IV).

The invention further provides a method of treating a condition, disease or disorder associated with inflammation, the method comprising administering to a subject in need thereof a therapeutically effective amount of at least one compound as disclosed herein above and below of general formula (I), (II), (III) and (IV).

In some embodiments, said medical condition, disease or disorder associated with inflammation is selected from the group consisting of multiple sclerosis rheumatoid arthritis, inflammatory bowel disease (IBD), Crohn's disease, ulcerative colitis, irritable bowel syndrome (IBS), systemic lupus erythematosus (SLE), cutaneous lupus erythematosus, psoriasis, Type I diabetes (IDDM), Sjogren's disease, autoimmune thyroid disease, acquired immunodeficiency syndrome (AIDS), sarcoidosis, autoimmune uveitis, autoimmune hepatitis, hypersensitivity lung diseases, hypersensitivity pneumonitis, delayed-type hypersensitivity, interstitial lung disease (ILD) (e.g., idiopathic pulmonary fibrosis, or ILD associated with rheumatoid arthritis or other inflammatory diseases); scleroderma; dermatitis (including atopic dermatitis and eczematous dermatitis), iritis, conjunctivitis, keratoconjunctivitis, idiopathic bilateral progressive sensorineural hearing loss, aplastic anemia, pure red cell anemia, idiopathic thrombocytopenia, polychondritis, Graves ophthalmopathy, amyotrophic lateral sclerosis (ALS), primary biliary cirrhosis, ileitis, chronic inflammatory intestinal disease, celiac disease, Alzheimers's disease, prion associated disease and cancer metastases.

In some embodiments, said medical condition, disease or disorder is selected from the group consisting of rheumatoid arthritis, atherosclerosis, Crohn's disease and multiple sclerosis.

In some embodiments, said treatment with at least one compound of the invention is a non-abusive treatment.

The invention further provides a method of treating pain in a subject in need thereof, the method comprising administering to a subject in need thereof a therapeutically effective amount of at least one compound as disclosed herein above and below of general formula (I), (II) and (III).

When referring to “pain” it should be understood to refer to any type of pain including transitory, lasting, chronic, acute, idiopathic pain and so forth. The pain to be treated may be caused by any condition, disease or disorder such as for example only until the noxious stimulus is removed or the underlying damage or pathology has healed, but some painful conditions, such as rheumatoid arthritis, peripheral neuropathy, cancer, any type of trauma, surgery, inflammation, child birth, and so forth.

The invention further provides a method of treating a condition, disease or disorder associated with obesity, the method comprising administering to a subject in need thereof a therapeutically effective amount of at least one compound as disclosed herein above and below of general formula (I), (II), (III) and (IV).

The present invention provides a method of treating a condition, disease or disorder associate with inflammation, pain, obesity and any combinations thereof; wherein said method comprises administering to a subject in need thereof at least one compound of general formula (I), including any stereoisomer, solvate or salt thereof:

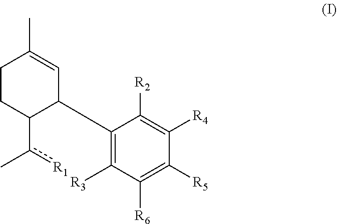

Wherein [symbol] is a single or double bond; R1 is selected from CH3 and CH2;

R2 and R3 are selected from OH, O(C1-C3 alkyl), straight or branched C3-C10 alkyl; R4 is a straight or branched C1-C5 alkyl; R5 is selected from OH, straight or branched C3-C10 alkyl; R6 is selected from H, straight or branched C1-C5 alkyl.

The present invention provides a method of treating a condition, disease or disorder associate with inflammation, pain, obesity and any combinations thereof; wherein said method comprises administering to a subject in need thereof at least one compound of general formula (II), including any stereoisomer, solvate or salt thereof:

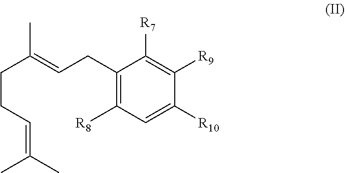

Wherein R7 and R8 are each selected from OH, O(C1-C3 alkyl), OC(═O)R16; R9 is selected from H, or C1-C3 alkyl; R10 is a straight or branched C3-C10 alkyl; R16 is C1-C5 alkyl optionally substituted by at least one of morpholino by at least one of morpholino, (O(CH2CH2)2NH) derivative.

The present invention provides a method of treating a condition, disease or disorder associate with inflammation, pain, obesity and any combinations thereof; wherein said method comprises administering to a subject in need thereof at least one compound of general formula (III), including a salt, solvate or stereoisomer thereof:

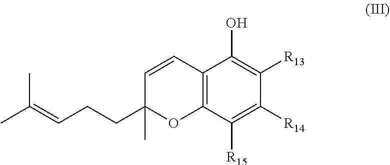

Wherein R13, R14 and R15 are each selected from H, straight or branched C1-C10 alkyl.

The present invention provides a method of treating a condition, disease or disorder associate with inflammation, pain, obesity and any combinations thereof; wherein said method comprises administering to a subject in need thereof at least one compound of general formula (IV), including a salt, solvate or stereoisomer thereof:

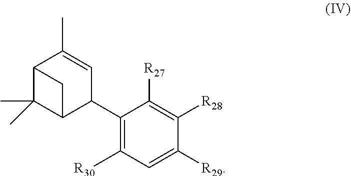

Where R27 and R30 are each selected from OH, O(C1-C3 alkyl); R28 and R29 are each selected from a straight or branched C1-C10 alkyl.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1A-1B shows the results of swelling (1A) and pain (1B) of HUM-216 in varied doses compared to CBD.

FIG. 2A-2B is shows the results of swelling (2A) and pain (2B) of HUM-223 and HUM-219 in varied doses compared to CBD and CBG.

FIG. 3A-3B is shows the results of TNF-alpha of HUM-216 in varied doses compared to CBD and of HUM-218 and HUM-223 in varied doses compared to CBG.

FIG. 4 shows the change in weight bearing of diseased knee as compared between CBD and HUM-216. High volume means more pain, low volume means less pain.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

The present embodiments further encompass any stereoisomers, prodrugs, solvates, hydrates and/or pharmaceutically acceptable salts of the compounds described herein.

As used herein, the term “stereoisomer” refers to isomers that possess identical constitution, but which differ in the arrangement of their atoms in space. In the context of the present invention this term includes enantiomers, diastereomers, racemates, non-racemic mixtures, (+)/(−)-isomers, (D)/(L)-isomers, (R)/(S)-isomers, cis-trans isomers, E/Z-isomers, and any combinations thereof.

The term “prodrug” refers to an agent, which is converted into the active compound (the active parent drug) in vivo. Prodrugs are typically useful for facilitating the administration of the parent drug. They may, for instance, be bioavailable by oral administration whereas the parent drug is not. A prodrug may also have improved solubility as compared with the parent drug in pharmaceutical compositions. Prodrugs are also often used to achieve a sustained release of the active compound in vivo. An example, without limitation, of a prodrug would be a compound as described herein, having one or more carboxylic acid moieties, which is administered as an ester (the “prodrug”). Such a prodrug is hydrolyzed in vivo, to thereby provide the free compound (the parent drug). The selected ester may affect both the solubility characteristics and the hydrolysis rate of the prodrug.

The term “solvate” refers to a complex of variable stoichiometry (e.g., di-, tri-, tetra-, penta-, hexa-, and so on), which is formed by a solute (the compound of the present invention) and a solvent, whereby the solvent does not interfere with the biological activity of the solute. Suitable solvents include, for example, ethanol ,acetic acid and the like.

The term “hydrate” refers to a solvate, as defined hereinabove, where the solvent is water.

The phrase “pharmaceutically acceptable salt” refers to a charged species of the parent compound and its counter ion, which is typically used to modify the solubility characteristics of the parent compound and/or to reduce any significant irritation to an organism by the parent compound, while not abrogating the biological activity and properties of the administered compound. An example, without limitation, of a pharmaceutically acceptable salt would be a carboxylate anion and a cation such as, but not limited to, ammonium, sodium, potassium and the like.

As is well known in the art, the phrase “acid addition salt” describes a complex of two ionizable moieties, a base and an acid, which, when interacted in a particular stoichiometric proportion and under suitable conditions, form a salt that comprises one or more cations of the base moiety and one or more anions of the acid moiety. As used herein, the phrase “acid addition salt” refers to such a complex, in which the base moiety in amine, such that the salt comprises a cationic form of the amine (ammonium) and an anionic form of an acid.

Depending on the stochiometric proportions between the base and the acid in the salt complex, as is detailed hereinbelow, the acid additions salts can be either mono addition salts or poly addition salts.

The phrase “mono addition salt”, as used herein, refers to a salt complex in which the stochiometric ratio between the acid anion and amine cation is 1:1, such that the acid addition salt includes one molar equivalent of the acid per one molar equivalent of the conjugate.

The phrase “poly addition salt”, as used herein, refers to a salt complex in which the stochiometric ratio between the acid anion and the amine cation is greater than 1:1 and is, for example, 2:1, 3:1, 4:1 and so on, such that the acid addition salt 15 includes two or more molar equivalents of the acid per one molar equivalent of the conjugate.

The stoichiometric proportions between the base and the acid of the salt complex, according to some embodiments of the present invention, ranges from 6:1 to 1:6 base:acid equivalents, from 4:1 to 1:4 base:acid equivalents, from 3:1 to 1:3 base:acid equivalents or from 1:1 to 1:3 base:acid equivalents.

The acid addition salts of a chemical conjugate according to the present invention are therefore complexes formed between one or more amino groups of the compound and one or more equivalents of an acid. The acid addition salts may therefore include a variety of organic and inorganic acids, such as, but not limited to, halogen acids such as hydrochloric acid which affords an hydrochloric acid addition salt (as well as salts of bromide and iodide), acetic acid which affords an acetic acid addition salt, ascorbic acid which affords an ascorbic acid addition salt, benzoic acid which affords a benzoic acid addition salt (benzoate), benzenesulfonic acid which affords a benzenesulfonic acid addition salt, camphorsulfonic acid which affords a camphorsulfonic acid addition salt, naphthylsulfonic acid which affords a naphthylsulfonic acid addition salt, toluenelsulfonic acid (p-toluenesulfonic acid) which affords a toluenesulfonic acid addition salt (tosylate), trifluoroacetic acid which affords a trifluoroacetic acid addition salt, citric acid which affords a citric acid addition salt, maleic acid which affords a maleic acid addition salt (maleate), methanesulfonic acid which affords a methanesulfonic acid (mesylate or methanesulfonate) addition salt, naphthalenesulfonic acid which affords a napsylate addition salt, oxalic acid which affords an oxalic acid addition salt, phosphoric acid which affords a phosphoric acid addition salt, succinic acid which affords a succinic acid addition salt (succinate), sulfuric acid which affords a sulfuric acid addition salt and tartaric acid which affords a tartaric acid addition salt. Each of these acid addition salts can be either a mono acid addition slat or a poly acid addition salt, as these terms are defined hereinabove.

As used herein, the term “alkyl” describes an aliphatic hydrocarbon including straight chain and branched chain groups. According to some embodiments, the alkyl group has 1 to 10 carbon atoms, according to other embodiments, 1 to 5 carbon atoms, according to yet other embodiments, 6 to 10 carbon atoms, and according to still other embodiments, 4 to 6 carbon atoms. Whenever a numerical range; e.g., “1-10”, is stated herein, it implies that the group, in this case the alkyl group, may contain 1 carbon atom, 2 carbon atoms, 3 carbon atoms, etc., up to and including 10 carbon atoms. The alkyl can be substituted or unsubstituted. When substituted, the substituent can be, for example, an alkyl, an alkenyl, an alkynyl, a cycloalkyl, an aryl, a heteroaryl, a halogen (halo), a hydroxy, an oxo, an alkoxy, an aryloxy, a thiohydroxy, a thioalkoxy, a thioaryloxy, a haloalkyl, an amine, a carbonyl, a carboxyl, an amide, a thioamide, a cyano and a carbamate, as these terms are defined herein.

The term “alkyl”, as used herein, also encompasses saturated or unsaturated hydrocarbon, hence this term further encompasses alkenyl and alkynyl.

The term “alkenyl” describes an unsaturated alkyl, as defined herein, having at least two carbon atoms and at least one carbon-carbon double bond. The alkenyl may be unsubstituted or substituted by one or more substituents, as described hereinabove for alkyl.

The term “alkynyl”, as defined herein, is an unsaturated alkyl having at least two carbon atoms and at least one carbon-carbon triple bond. The alkynyl may be unsubstituted or substituted by one or more substituents, as described hereinabove for alkyl.

As used herein, the term “amine” describes a —NR′R″ group where each of R′ and R″ is independently hydrogen, alkyl, cycloalkyl, heteroalicyclic, aryl or heteroaryl, as these terms are defined herein.

As used herein, the terms “halo”, “halogen” and “halide”, which are referred to herein interchangeably, describe an atom of a fluorine, chlorine, bromine or iodine, also referred to herein as fluoride, chloride, bromide and iodide.

The term “haloalkyl” describes an alkyl group as defined above, further substituted by one or more halide(s).

The term “hydroxy” or “hydroxyl”, as used herein interchangeably, refers to an —OH group.

The term “alkoxy” describes a -OR' group, where R′ is as defined herein.

The term “aryloxy”, as used herein, refers to an —OR″ group wherein R″ is aryl.

The term “thiohydroxy”, as used herein, refers to an —SH group.

The term “thioalkoxy” describes a —SR′ group, where R′ is as defined herein.

The term “thioarylkoxy” describes a —SR″ group, where R″ is aryl.

The term “carbonyl”, or “ketone”, as used herein, refers to —(C═O)H or —(C═O)—R group, wherein R is as defined herein. An exemplary carbonyl is a formyl group, wherein R′ is hydrogen. Another exemplary carbonyl is an acetyl group, wherein R′ is methyl.

The term “oxo” refers to a (═O) group, namely an oxygen bound by a double bond, which in the case of a carbon substituent constitutes a carbonyl.

The terms “carboxy”, “carboxyl” or “carboxylate”, as used herein, refer interchangeably to a —C(═O)—O—R, where R can be absent (as in the case of a carboxylate anion), or selected from the group consisting of hydrogen (for example, carboxylic acid), alkyl (for example, ester), cycloalkyl, heteroalicyclic, aryl or heteroaryl, as these terms are defined herein.

The term “amide describes a —C(═O)—NRR”, where R is as defined herein

The term “thioamide” describes a —C(═S)—NRR″, where R is as defined herein

The term “thioimide” describes a —C(═NR)—SR″, where R and R″ are as defined herein.

The term “cyano”, as used herein, refers to a —C═N group.

The term “carbamate” describes an —OC(═O)—NR′R″, with R′ and R″ as defined herein.

A particular exemplary carbamate is afforded when an amine is protected with a Boc protecting group, affording a tert-butyl carbamate.

Another exemplary carbamate is afforded when an amine is protected with an Fmoc protecting group, affording a (9H-fluoren-9-yl)methyl carbamate.

Further these cannabinoid derivative compounds are part of a group of compounds which include, for example:

(1′R,2′R)-3,5′-dimethyl-4-pentyl-2′-(prop-1-en-2-yl)-1′,2′,3′,4′-tetrahydro-[1,1′-biphenyl]-2,6-diol (HUM-216)

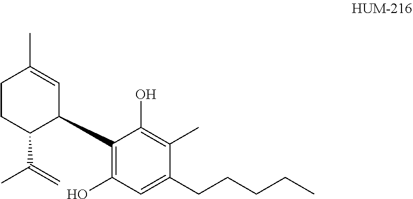 [SMILES: C=C(C)[C@@H]1CCC(C)=C[C@H]1c1c(O)cc(CCCCC)c(C)c1O]

1′S,2′S)-2′-isopropyl-3,5′-dimethyl-4-pentyl-1′,2′,3′,4′-tetrahydro-[1,1′-biphenyl]-2,6-diol (HUM-217)

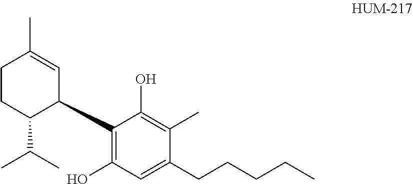 [SMILES: CCCCCc1cc(O)c([C@@H]2C=C(C)CC[C@H]2C(C)C)c(O)c1C]

(E)-2-(3,7-dimethylocta-2,6-dien-1-yl)-4-methyl-5-pentylbenzene-1,3-diol (HUM-218)

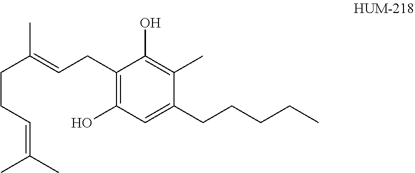 [SMILES: CCCCCc1cc(O)c(C/C=C(\C)CCC=C(C)C)c(O)c1C]

(1R,2R)-2′,6′-dimethoxy-3′,5-dimethyl-4′-pentyl-2-(prop-1-en-2-yl)-1,2,3,4-tetrahydro-1,1′-biphenyl (HUM-219)

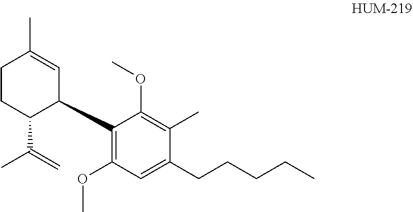 [SMILES: C=C(C)[C@@H]1CCC(C)=C[C@H]1c1c(OC)cc(CCCCC)c(C)c1OC]

(E)-2-(3,7-dimethylocta-2,6-dien-1-yl)-3-methoxy-5-(2-methyloctan-2-yl)phenol (HUM-223)

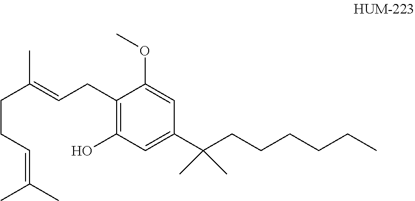 [SMILES: CCCCCCC(C)(C)c1cc(O)c(C/C=C(\C)CCC=C(C)C)c(OC)c1]

(1′R,2′R)-5,5′-dimethyl-6-pentyl-2′-(prop-1-en-2-yl)-1′,2′,3′,4′-tetrahydro-[1,1′-biphenyl]-2,4-diol (HUM-229)

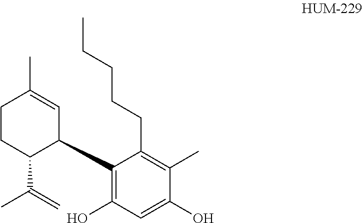 [SMILES: C=C(C)[C@@H]1CCC(C)=C[C@H]1c1c(O)cc(O)c(C)c1CCCCC]

(E)-4-(3,7-dimethylocta-2,6-dien-1-yl)-6-methyl-5-pentylbenzene-1,3-diol (HUM-330)

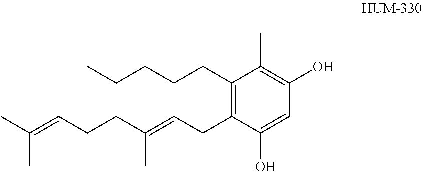 [SMILES: CCCCCc1c(C)c(O)cc(O)c1C/C=C(\C)CCC=C(C)C]

4-methyl-5-pentyl-2-((2R)-4,6,6-trimethylbicyclo[3.1.1]hept-3-en-2-yl)benzene-1,3-diol (HUM-231)

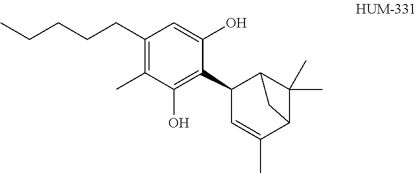 [SMILES: CCCCCc1cc(O)c([C@@H]2C=C(C)C3CC2C3(C)C)c(O)c1C]

2,6-dimethyl-2-(4-methylpent-3-en-1-yl)-7-pentyl-2H-chromen-5-ol

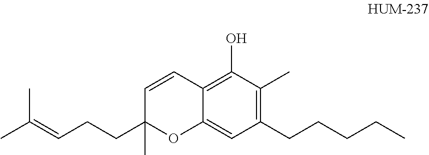 [SMILES: CCCCCc1cc2c(c(O)c1C)C=CC(C)(CCC=C(C)C)O2]

(E)-4-(3-(2-(3,7-dimethylocta-2,6-dien-1-yl)-3-methoxy-5-pentylphenoxy)-3-oxopropyl)morpholin-4-ium-3-ide (HUM-233)

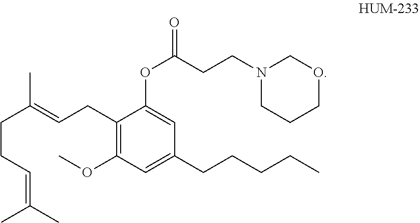 [SMILES: CCCCCc1cc(OC)c(C/C=C(\C)CCC=C(C)C)c(OC(=O)CCN2CCCOC2)c1]

Thus, according to another aspect of the present invention, there is provided a method of treating a medical condition, a disease or a disorder associated with inflammation, which is affected by administering to a subject in need thereof a therapeutically effective amount of one or more of the compounds as presented herein, as well enantiomers, hydrates, solvates, prodrugs or any pharmaceutically acceptable salts thereof, as defined hereinabove.

According to some embodiments of the present invention, there is provided a method of treating a medical condition, a disease or a disorder associated with inflammation, which is effected by administering to a subject in need thereof a therapeutically effective amount of one or more of any one of the compounds presented in Table I which is presented in the Examples section that follows hereinbelow, as well enantiomers, hydrates, solvates, prodrugs or any pharmaceutically acceptable salts thereof, as defined hereinabove.

As used herein, the terms “treating”, and “treatment” includes abrogating, substantially inhibiting, slowing or reversing the progression of a condition, substantially ameliorating clinical or aesthetical symptoms of a condition or substantially preventing the appearance of clinical or aesthetical symptoms of a condition.

Accordingly, another aspect of the present invention provides a use of one or more of the compounds as presented herein, as well enantiomers, hydrates, solvates, prodrugs or any pharmaceutically acceptable salts thereof, as defined hereinabove, in the preparation of a medicament.

According to some embodiments of the present invention, the medicament is for the treatment of a medical condition, a disease or a disorder associated with inflammation.

According to some embodiments of the present invention, there is provided a use of one or more of any one of the compounds presented in Table I which is presented in the Examples section that follows hereinbelow, as well as any enantiomers, hydrates, solvates, prodrugs or any pharmaceutically acceptable salts thereof, as defined hereinabove, in the preparation of a medicament for the treatment of a medical condition, a disease or a disorder associated with inflammation.

Inflammation is a protective response of the body to an injury. As used herein, the term “inflammation” includes without limitation, medical conditions, diseases and disorders which are associated with inflammation.

Thus, representative examples of diseases or disorders associated with inflammation, and are therefore treatable by using one or more of the compounds described in the present invention include, without limitation, idiopathic inflammatory diseases or disorders, chronic inflammatory diseases or disorders, acute inflammatory diseases or disorders, autoimmune diseases or disorders, infectious diseases or disorders, inflammatory malignant diseases or disorders, inflammatory transplantation-related diseases or disorders, inflammatory degenerative diseases or disorders, diseases or disorders associated with a hypersensitivity, inflammatory cardiovascular diseases or disorders, inflammatory cerebrovascular diseases or disorders, peripheral vascular diseases or disorders, inflammatory glandular diseases or disorders, inflammatory gastrointestinal diseases or disorders, inflammatory cutaneous diseases or disorders, inflammatory hepatic diseases or disorders, inflammatory neurological diseases or disorders, inflammatory musculo-skeletal diseases or disorders, inflammatory renal diseases or disorders, inflammatory reproductive diseases or disorders, inflammatory systemic diseases or disorders, inflammatory connective tissue diseases or disorders, inflammatory tumors, necrosis, inflammatory implant-related diseases or disorders, inflammatory aging processes, immunodeficiency diseases or disorders, proliferative diseases and disorders, such as cancer, and inflammatory pulmonary diseases or disorders, as is detailed hereinbelow.

Non-limiting examples of hypersensitivities include Type I hypersensitivity, Type II hypersensitivity, Type III hypersensitivity, Type IV hypersensitivity, immediate hypersensitivity, antibody mediated hypersensitivity, immune complex mediated hypersensitivity, T lymphocyte mediated hypersensitivity, delayed type hypersensitivity, helper T lymphocyte mediated hypersensitivity, cytotoxic T lymphocyte mediated hypersensitivity, TH1 lymphocyte mediated hypersensitivity, and TH2 lymphocyte mediated hypersensitivity.

Non-limiting examples of inflammatory cardiovascular disease or disorder include occlusive diseases or disorders, atherosclerosis, a cardiac valvular disease, stenosis, restenosis, in-stent-stenosis, myocardial infarction, coronary arterial disease, acute coronary syndromes, congestive heart failure, angina pectoris, myocardial ischemia, thrombosis, Wegener' s granulomatosis, Takayasu's arteritis, Kawasaki syndrome, anti-factor VIII autoimmune disease or disorder, necrotizing small vessel vasculitis, microscopic polyangiitis, Churg and Strauss syndrome, pauci-immune focal necrotizing glomerulonephritis, crescentic glomerulonephritis, antiphospholipid syndrome, antibody induced heart failure, thrombocytopenic purpura, autoimmune hemolytic anemia, cardiac autoimmunity, Chagas' disease or disorder, and anti-helper T lymphocyte autoimmunity.

Stenosis is an occlusive disease of the vasculature, commonly caused by atheromatous plaque and enhanced platelet activity, most critically affecting the coronary vasculature.

Restenosis is the progressive re-occlusion often following reduction of occlusions in stenotic vasculature. In cases where patency of the vasculature requires the mechanical support of a stent, in-stent-stenosis may occur, re-occluding the treated vessel.

Non-limiting examples of cerebrovascular diseases or disorders include stroke, cerebrovascular inflammation, cerebral hemorrhage and vertebral arterial insufficiency.

Non-limiting examples of peripheral vascular diseases or disorders include gangrene, diabetic vasculopathy, ischemic bowel disease, thrombosis, diabetic retinopathy and diabetic nephropathy.

Non-limiting examples of autoimmune diseases or disorders include all of the diseases caused by an immune response such as an autoantibody or cell-mediated immunity to an autoantigen and the like. Representative examples are chronic rheumatoid arthritis, juvenile rheumatoid arthritis, systemic lupus erythematosus, scleroderma, mixed connective tissue disease, polyarteritis nodosa, polymyositis/dermatomyositis, Sjogren's syndrome, Bechet's disease, multiple sclerosis, autoimmune diabetes, Hashimoto's disease, psoriasis, primary myxedema, pernicious anemia, myasthenia gravis, chronic active hepatitis, autoimmune hemolytic anemia, idiopathic thrombocytopenic purpura, uveitis, vasculitides and heparin induced thrombocytopenia.

Non-limiting examples of inflammatory glandular diseases or disorders include pancreatic diseases or disorders, Type I diabetes, thyroid diseases or disorders, Graves' disease, thyroiditis, spontaneous autoimmune thyroiditis, Hashimoto's thyroiditis, idiopathic myxedema, ovarian autoimmunity, autoimmune anti-sperm infertility, autoimmune prostatitis and Type I autoimmune polyglandular syndrome.

Non-limiting examples of inflammatory gastrointestinal diseases or disorders include colitis, ileitis, Crohn's disease, chronic inflammatory intestinal disease, inflammatory bowel syndrome, chronic inflammatory bowel disease, celiac disease, ulcerative colitis, an ulcer, a skin ulcer, a bed sore, a gastric ulcer, a peptic ulcer, a buccal ulcer, a nasopharyngeal ulcer, an esophageal ulcer, a duodenal ulcer and a gastrointestinal ulcer.

Non-limiting examples of inflammatory cutaneous diseases or disorders include acne, and an autoimmune bullous skin disease.

Non-limiting examples of inflammatory hepatic diseases or disorders include autoimmune hepatitis, hepatic cirrhosis, and biliary cirrhosis.

Non-limiting examples of inflammatory neurological diseases or disorders include multiple sclerosis, Alzheimer's disease, Parkinson's disease, myasthenia gravis, motor neuropathy, Guillain-Barre syndrome, autoimmune neuropathy, Lambert-Eaton myasthenic syndrome, paraneoplastic neurological disease or disorder, paraneoplastic cerebellar atrophy, non-paraneoplastic stiff man syndrome, progressive cerebellar atrophy, Rasmussen's encephalitis, amyotrophic lateral sclerosis, Sydeham chorea, Gilles de la Tourette syndrome, autoimmune polyendocrinopathy, dysimmune neuropathy, acquired neuromyotonia, arthrogryposis multiplex, Huntington's disease, AIDS associated dementia, amyotrophic lateral sclerosis (AML), multiple sclerosis, stroke, an inflammatory retinal disease or disorder, an inflammatory ocular disease or disorder, optic neuritis, spongiform encephalopathy, migraine, headache, cluster headache, and stiff-man syndrome.

Non-limiting examples of inflammatory connective tissue diseases or disorders include autoimmune myositis, primary Sjogren's syndrome, smooth muscle autoimmune disease or disorder, myositis, tendinitis, a ligament inflammation, chondritis, a joint inflammation, a synovial inflammation, carpal tunnel syndrome, arthritis, rheumatoid arthritis, osteoarthritis, ankylosing spondylitis, a skeletal inflammation, an autoimmune ear disease or disorder, and an autoimmune disease or disorder of the inner ear.

Non-limiting examples of inflammatory renal diseases or disorders include autoimmune interstitial nephritis and/or renal cancer.

Non-limiting examples of inflammatory reproductive diseases or disorders include repeated fetal loss, ovarian cyst, or a menstruation associated disease or disorder.

Non-limiting examples of inflammatory systemic diseases or disorders include systemic lupus erythematosus, systemic sclerosis, septic shock, toxic shock syndrome, and cachexia.

Non-limiting examples of infectious disease or disorder include chronic infectious diseases or disorders, a subacute infectious disease or disorder, an acute infectious disease or disorder, a viral disease or disorder, a bacterial disease or disorder, a protozoan disease or disorder, a parasitic disease or disorder, a fungal disease or disorder, a mycoplasma disease or disorder, gangrene, sepsis, a prion disease or disorder, influenza, tuberculosis, malaria, acquired immunodeficiency syndrome, and severe acute respiratory syndrome.

Non-limiting examples of inflammatory transplantation-related diseases or disorders include graft rejection, chronic graft rejection, subacute graft rejection, acute graft rejection hyperacute graft rejection, and graft versus host disease or disorder. Exemplary implants include a prosthetic implant, a breast implant, a silicone implant, a dental implant, a penile implant, a cardiac implant, an artificial joint, a bone fracture repair device, a bone replacement implant, a drug delivery implant, a catheter, a pacemaker, an artificial heart, an artificial heart valve, a drug release implant, an electrode, and a respirator tube.

Non-limiting examples of inflammatory tumors include a malignant tumor, a benign tumor, a solid tumor, a metastatic tumor and a non-solid tumor.

Non-limiting examples of inflammatory pulmonary diseases or disorders include asthma, allergic asthma, emphysema, chronic obstructive pulmonary disease or disorder, sarcoidosis and bronchitis.

Exemplary medical conditions, diseases and disorders which are associated with inflammation, according to some embodiments of the present invention include multiple sclerosis rheumatoid arthritis, inflammatory bowel disease (IBD), Crohn's disease, ulcerative colitis, irritable bowel syndrome (IBS), systemic lupus erythematosus (SLE), cutaneous lupus erythematosus, psoriasis, Type I diabetes (IDDM), Sjogren's disease, autoimmune thyroid disease, acquired immunodeficiency syndrome (AIDS), sarcoidosis, autoimmune uveitis, autoimmune hepatitis, hypersensitivity lung diseases, hypersensitivity pneumonitis, delayed-type hypersensitivity, interstitial lung disease (ILD) (e.g., idiopathic pulmonary fibrosis, or ILD associated with rheumatoid arthritis or other inflammatory diseases; (scleroderma; dennatitis (including atopic dermatitis and eczematous dermatitis), iritis, conjunctivitis, keratoconjunctivitis, idiopathic bilateral progressive sensorineural hearing loss, aplastic anemia, pure red cell anemia, idiopathic thrombocytopenia, polychondritis, Graves ophthalmopathy, amyotrophic lateral sclerosis (ALS), primary biliary cirrhosis, ileitis, chronic inflammatory intestinal disease, celiac disease, Alzheimers's disease, prion associated disease and cancer metastases.

According to some embodiments of the present invention, the inflammatory disorders include rheumatoid arthritis, atherosclerosis, Crohn's disease, multiple sclerosis, Alzheimers's disease, prion associated disease and cancer metastases.

As used herein, the phrase “therapeutically effective amount” describes an amount of the compound being administered which will relieve to some extent one or more of the symptoms of the condition being treated.

As demonstrated in the examples section that follows, an exemplary therapeutically effective amount of the compounds of the present invention ranges between about 0.1 mg/kg body and about 100 mg/kg body.

In any of the methods and uses described herein, the cannabinoid derivative compounds of the present embodiments can be utilized either per se or, according to some embodiments, as a part of a pharmaceutical composition that further comprises a pharmaceutically acceptable carrier.

Thus, according to additional aspects of the present invention, there is provided a pharmaceutical composition, which comprises one or more compounds having general Formula I, as defined hereinabove, and a pharmaceutically acceptable carrier.

According to some embodiments of the present invention, there is provided a pharmaceutical composition, which comprises one or more of any one of the compounds presented in Table I which is presented in the Examples section that follows hereinbelow, and a pharmaceutically acceptable carrier, as well as any enantiomers, hydrates, solvates, prodrugs or any pharmaceutically acceptable salts thereof, as defined hereinabove.

As used herein a “pharmaceutical composition” refers to a preparation of the compounds presented herein, with other chemical components such as pharmaceutically acceptable and suitable carriers and excipients. The purpose of a pharmaceutical composition is to facilitate administration of a compound to an organism.

Hereinafter, the term “pharmaceutically acceptable carrier” refers to a carrier or a diluent that does not cause significant irritation to an organism and does not abrogate the biological activity and properties of the administered compound. Examples, without limitations, of carriers are propylene glycol, saline, emulsions and mixtures of organic solvents with water, as well as solid (e.g., powdered) and gaseous carriers.

Herein the term “excipient” refers to an inert substance added to a pharmaceutical composition to further facilitate administration of a compound. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington' s Pharmaceutical Sciences” Mack Publishing Co., Easton, PA, latest edition, which is incorporated herein by reference.

Pharmaceutical compositions for use in accordance with the present invention thus may be formulated in conventional manner using one or more pharmaceutically acceptable carriers comprising excipients and auxiliaries, which facilitate processing of the compounds into preparations which can be used pharmaceutically. Proper formulation is dependent upon the route of administration chosen. The dosage may vary depending upon the dosage form employed and the route of administration utilized. The exact formulation, route of administration and dosage can be chosen by the individual physician in view of the patient's condition (see e.g., Fingl et al., 1975, in “The Pharmacological Basis of Therapeutics”, Ch. I p. I.(The pharmaceutical composition may be formulated for administration in either one or more of routes depending on whether local or systemic treatment or administration is of choice, and on the area to be treated. Administration may be done orally, by inhalation, or parenterally, for example by intravenous drip or intraperitoneal, subcutaneous, intramuscular or intravenous injection, or topically (including ophtalmically, vaginally, rectally, intranasally).

Formulations for topical administration may include but are not limited to lotions, ointments, gels, creams, suppositories, drops, liquids, sprays and powders.

Conventional pharmaceutical carriers, aqueous, powder or oily bases, thickeners and the like may be necessary or desirable.

Compositions for oral administration include powders or granules, suspensions or solutions in water or non-aqueous media, sachets, pills, caplets, capsules or tablets. Thickeners, diluents, flavorings, dispersing aids, emulsifiers or binders may be desirable.

Formulations for parenteral administration may include, but are not limited to, sterile solutions which may also contain buffers, diluents and other suitable additives. Slow release compositions are envisaged for treatment.

The amount of a composition to be administered will, of course, be dependent on the subject being treated, the severity of the affliction, the manner of administration, the judgment of the prescribing physician, etc.

Compositions of the present invention may, if desired, be presented in a pack or dispenser device, such as an FDA (the U.S. Food and Drug Administration) approved kit, which may contain one or more unit-dosage forms containing the active ingredient. The pack may, for example, comprise metal or plastic foil, such as, but not limited to a blister pack or a pressurized container (for inhalation). The pack or dispenser device may be accompanied by instructions for administration. The pack or dispenser may also be accompanied by a notice associated with the container in a form prescribed by a governmental agency regulating the manufacture, use or sale of pharmaceuticals, which notice is reflective of approval by the agency of the form of the compositions for human or veterinary administration. Such notice, for example, may be of labeling approved by the U.S. Food and Drug Administration for prescription drugs or of an approved product insert.

Compositions comprising a compound of the invention formulated in a compatible pharmaceutical carrier may also be prepared, placed in an appropriate container, and labeled for treatment of a medical condition, disease or disorder associated with inflammation, as is detailed hereinabove.

Thus, according to an embodiment of the present invention, the pharmaceutical composition of the present invention is being packaged in a packaging material and identified in print, in or on the packaging material, for use in the treatment of a medical condition, disease or disorder associated with inflammation, as is defined hereinabove.

According to further embodiments of the any of the methods, uses and compositions presented herein, the compounds of the present invention can be combined with other active ingredients which are commonly used to treat inflammation-associated diseases and disorders.

When referring to “non-abusive treatment” it should be understood to relate to the treatment with at least one compound which does not subject a patient administered with said compound to substance abuse, drug abuse, in any amount administered, by methods which are harmful to the individual or others.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions; illustrate the invention in a non-limiting fashion.

### Example 1 Chemical Syntheses

### Materials and Methods:

AU chemical reagents were purchased from Sigma-Aldrich.

Organic solvents were purchased from Bio-Lab.

Cannabinoids (such as cannabidiol) were extracted from Cannabis sativa plant as previously described [Gaoni, Y. and Mechoulam, R. (1971) J. Amer. Chem. Soc. 93, 217-224].

### Standard Carboxylation Reaction

Based on procedure by Ben-Zvi et al [26]. 20 mmol of the cannabinoid compound were dissolved in 30 mL of 2M Magnesium Methyl Carbonate (MMC) solution in DMF (60 mmol). A condenser was attached, and the reaction was heated to 120° C. for 2 hours, monitoring by TLC.

The reaction was worked up by pouring it into ice-cold 10% HCl solution w/v. The aqueous phase was extracted three times with diethyl ether (Et₂O). The organic phase was then dried over anhydrous Magnesium Sulphate (MgSO₄) and evaporated to give dark purple syrupy crude. The CBDA was purified by silica gel column chromatography using Ethyl Acetate (EA):Methanol:Acetic Acid (10%:2%:1% respectively) in Petroleum Ether (approximate yield: 35%).

### Standard Reduction of Carboxylic Acids to Methyl Group

Based on methods previously published by H. Edery et al [27]. 3.04 mmol of carboxylic acid, were dissolved in dry THF under nitrogen. The solution was then slowly added to a flask containing a pre-cooled suspension of 79.01 mmol of LiAlH₄ in dry THF (30 mL, cooled to −20° C.). A condenser was attached, and the reaction was heated to reflux for three hours, monitoring by TLC. Upon full consumption of starting material, the reaction was cooled to −20° C. for the workup. Ethyl acetate was added drop wise to neutralize the remaining LiAlH₄, followed by ethanol, methanol and ice until there is no visible reaction of the LiAlH₄ in the flask. The reaction suspension was washed with 10% w/v HCl solution to pH 1. The aqueous phase was extracted three times with ethyl acetate, washed with sat. NaHCO₃ to pH 10 and brine to neutral pH. The organic phase was dried over MgSO₄ and evaporated. The crude product was purified by silica gel column chromatography (approximate yield 95%).

H2-CBD Preparation: 5 grams (15.92 mmol) of Cannabidiol and 250 mg (1.1 mmol, 5% w/w) of Platinum Oxide were added to a pressure resistance flask and dissolved in ethyl acetate. The reaction flask was then vacuumed and pressurized with hydrogen gas to 15 psi. The reaction flask was placed on an automatic shaker and the pressure was maintained for 1.5 minutes. The hydrogen was then removed by vacuum. The mixture was gravity filtered and evaporated. The crude product was purified by silica gel column chromatography (0 to 3% Et2O in petroleum ether)

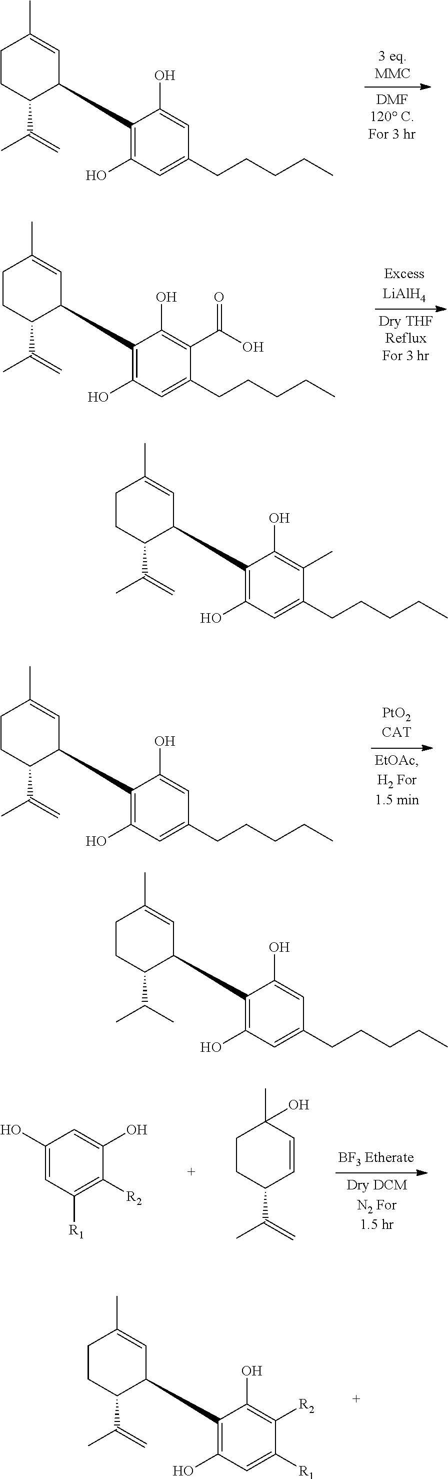

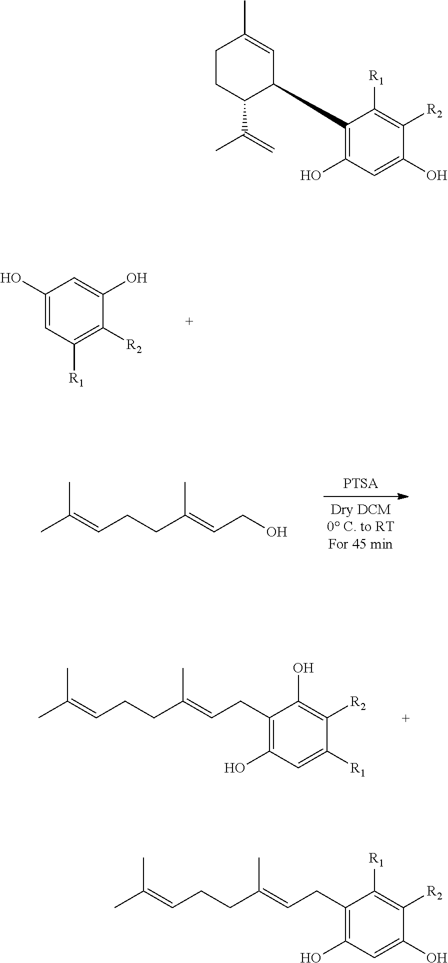

The synthesis of HUM-216: 2 g of LiAlH₄ were portionally added to 100 ml of dry THF at −15° C. at the nitrogen atmosphere, then 700 mg of CBDA dissolved in 50 ml dry THF was slowly added. The reaction was allowed to warm to room temperature, then refluxed for 5 hours. Then the reaction was cooled to −15° C. again, ethyl acetate was slowly dropped inside, until LiAlH₄ stops reacting, then a few drops of ethanol, methanol and water, to ensure that LiAlH₄ is not active any more. The product (98% yield) is extracted by 5 portions of ethyl acetate, washed to neutral, dried over MgSO4 and purified by silica gel chromatography with 5% diethyl ether in petroleum ether.

The synthesis of HUM-217: HUM-217 was prepared from the H2-CBD by the same way as HUM-216 from CBD, with quite the same yield (around 30%).

The preparation of CBG: To 2 g of olivetol dissolved in 200 ml dry dichloromethane 80 mg of para toluene sulfonic acid was added under N₂ atmosphere, the reaction was cooled to 0° C. 3 g of geraniol dissolved in 100 ml dry DCM was added slowly over the period of 10 min. The reaction is allowed to warm to room temp. and stirred 30 min. Then the reaction suspension was washed with 10% w/v NaHCO₃ solution to neutral, the aqueous phase was extracted three times with DCM and dried over MgSO₄. The product (30% yield) was purified by silica gel chromatography with 7% diethyl ether in petroleum ether.

The synthesis of HUM-218: HUM-218 was prepared from the CBG by the same way as HUM-216 from CBD, with quite the same yield (around 30%).

The synthesis of HUM-219: To 280 mg of HUM-216 (0.85 mmol) and 0.75 g K₂CO₃ in 10 ml dry DMF under N₂ atmosphere the excess of 500 ul of methyl iodide (˜7.5 mmol) was added dropwise. The reaction was stirred overnight. The reaction suspension was washed with 10% w/v HCl solution to neutral. The aqueous phase was extracted three times with ethyl acetate and dried over MgSO₄. The product (yield ˜90%) was purified by silica gel chromatography with 3% diethyl ether in petroleum ether.

The preparation of CBG-DMH: To 1.84 g of dimethylheptyl resorcinol (DMHR) dissolved in 100 ml dry dichloromethane 80 mg of para toluene sulfonic acid was added under N₂ atmosphere, the reaction was cooled to 0° C. 1.2 g of geraniol dissolved in 50 ml dry DCM was added slowly over the period of 10 min. The reaction is allowed to warm to room temp. and stirred 30 min. Then the reaction suspension was washed with 10% w/v NaHCO₃ solution to neutral, the aqueous phase was extracted three times with DCM and dried over MgSO₄. The product (30% yield) was purified by by silica gel chromatography with 5% diethyl ether in petroleum ether.

The synthesis of HUM-223: To 2.4 g of CBG-DMH and 1.34 g K₂CO₃ in 20 ml dry DMF under N₂ atmosphere, 406.1 ul of methyl iodide (1 eq) was added dropwise. The reaction was stirred overnight. The reaction suspension was washed with 10% w/v HCl solution to neutral. The aqueous phase was extracted three times with ethyl acetate and dried over MgSO₄. The product (yield ˜40%) was purified by silica gel chromatography with 3% diethyl ether in petroleum ether.

### Methods Developed During This Project

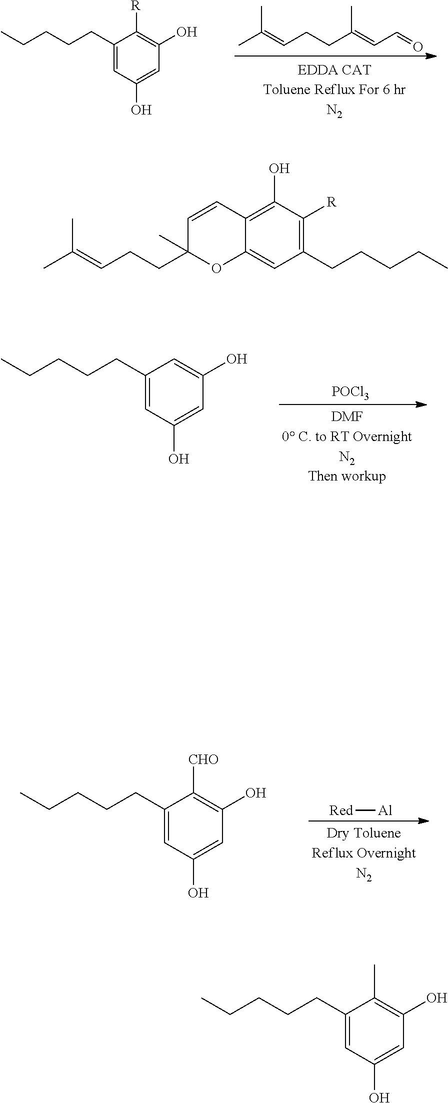

R═CHO or methyl.

Preparation of (+) CBC derivatives by citral coupling [28]: 0.5 gr of olivetol aldehyde (2.4 mmol) and 0.5 mL of citral (2.9 mmol) were dissolved in anhydrous toluene under nitrogen atmosphere at room temperature. 86 mg of Ethylendiamine Diacetate (EDDA) were added to the solution still at room temperature. A water condenser was then attached and the reaction was refluxed and monitored by TLC (5% EA in PE). After 4 hours the reaction was cooled to room temperature on an ice bath. Solvent was removed by vacuum evaporation to yield dark-orange oily crude. The product was purified by flash column chromatography using Et2O:PE gradient. Elution of the product at 1.25% Et2O. The product was obtained as a bright yellow oil.

Olivetol Formylation Reaction [29]: 15 mL of Phosphoryl Chloride (0.123 mol) were slowly and dropwise dissolved in an ice cold anhydrous DMF (43 mL) under nitrogen atmosphere. To it was added drop wise a 24 mL solution of 8.8 gr (0.05 mol) of olivetol in anhydrous DMF. The reaction was allowed to slowly warm to room temperature and was stirred overnight. The reaction was cooled on ice and to it was added drop wise 72 mL of ice cold water. While still on ice, 20% solution of Sodium Hydroxide was added to the reaction until pH of 10 was achieved. The reaction was then refluxed for 10 minutes. The reaction was them acidified using conc. Hydrochloric acid until pH of 1 was achieved. The aqueous reaction solution was extracted 4 times with ethyl acetate. The combined organic extractions were washed with brine, dried over magnesium sulfate and the solvent was evaporated on vacuum. The crude oil was dry loaded on silica gel and purified by column chromatography using EA:PE gradient. Product was eluted at 15% EA:PE. Starting material was eluted at 30% EA:PE. The product was obtained as pale-yellow solid. NMR spectrum (CDCl3) was in accordance with previously published literature.

Aldehyde reduction reaction [30]: 0.1 gr of aldehyde (0.48 mmol) were dissolved in anhydrous toluene under nitrogene atmosphere and cooled on an ice bath for 5 minutes. To the cold suspension was added 0.5 mL of 60% solution of Red-Al® in toluene. The reaction was then refluxed overnight. The reaction was cooled on an ice bath and 5 mL of brine were added drop wise to avoid spillage. The mixture was partitioned between ether and water and the aqueous phase was acidified to pH of 1 using 20% sulfuric acid solution. The now acidic aqueous phase was then extracted three timed with ether. The combined extractions were washed with sat. sodium bicarbonate and brine and dried over magnesium sulfate. The evaporated crude was purified by column chromatography using EA:PE gradient. Yield: 75%. NMR spectrum (CDCl3) was in accordance with previously published literature.

An alternative way for HUM-216 and the synthesis of the abnormal derivative HUM-229: 1.5 g of methyl-olivetol and 1.2 g of para-mentha dienol were dissolved in 50 ml dry DCM, 1 g of dry MgSO₄ was added under N₂ atmosphere. The reaction was cooled to 0° C., and 50 ul of BF₃ etherate were added. The reaction was mixed for 1.5 h, washed with 10% w/v NaHCO₃ solution to neutral, the aqueous phase was extracted three times with DCM and dried over MgSO₄. The crude was purified by by silica gel chromatography with 5%->30% diethyl ether in petroleum ether. HUM-216 was obtained at 45% yield, and HUM-229 at 35% yield.

An alternative way for HUM-218 and the synthesis of the abnormal derivative HUM-230: To 1.51 g of methyl-olivetol dissolved in 25 ml dry dichloromethane 140 mg of para toluene sulfonic acid was added under N₂ atmosphere, the reaction was cooled to 0° C. 1.2 g of geraniol dissolved in 20 ml dry DCM was added slowly over the period of 10 min. The reaction is allowed to warm to room temp. and stirred 30 min. Then the reaction suspension was washed with 10% w/v NaHCO₃ solution to neutral, the aqueous phase was extracted three times with DCM and dried over MgSO₄. The crude was purified by silica gel chromatography with 5%->30% diethyl ether in petroleum ether. HUM-218 was obtained at 45% yield, and HUM-229 at 30% yield.

The synthesis of HUM-231 and HUM-229: To 500 mg of methyl-olivetol dissolved in 70 ml dry dichloromethane 40 mg of para toluene sulfonic acid was added under N₂ atmosphere, the reaction was cooled to 0° C. 242 mg of verbenol dissolved in 15 ml dry DCM was added slowly over the period of 10 min. The reaction stirred at 0° C. for 1 h. Then the reaction suspension was washed with 10% w/v NaHCO₃ solution to neutral, the aqueous phase was extracted three times with DCM and dried over MgSO₄. The crude was purified by silica gel chromatography with 5%->30% diethyl ether in petroleum ether. HUM-231 was obtained at 45% yield, and HUM-229 at 25% yield.

### Example 2 Biological Activity

Preparation of murine macrophages for anti-inflammatory activity measurement assays: The anti-inflammatory activity of exemplary cannabidiol derivatives, according to some embodiments of the present invention, was tested using murine macrophages. Peritoneal exudate macrophages from 8-week-old to 9-week-old C57BL/6 female mice were harvested 4 days after injection of 1.5 ml of 3% thioglycolate medium. Peritoneal macrophages were cultured in 96-microwell flat bottomed plates. Two hours later, the cells were rinsed to remove unattached cells, and thereafter activation with LPS (1 pg/ml). The exemplary cannabinoid derivatives were diluted to various concentrations with DMEM and added to the cell preparations. After 24 hours incubation, the supernatants were collected and stored at −20° C. until assayed for TNF-α and NO.

Determination of plasma TNF-α levels in endotoxemic mice: The cannabidiol derivatives were dissolved in one-part DMSO, one part TWEEN 80, and four parts PBS, and controls were treated with the same vehicle. Male C57B16/J mice were pretreated with the indicated exemplary cannabidiol derivative 45 minutes prior to an intraperitoneal injection of 1 mg/kg LPS. After an additional 90 minutes, the mice were decapitated, and trunk blood was collected for determination of TNF-α levels.

Reactive oxygen intermediate (ROI) assay: RAW 264.7 cells, suspended in HBSS without phenol red, were distributed in plastic luminometer tubes. The cannabidiol derivatives were added to the samples followed by addition of 10 μl of luminol and 30 μl of zymosan. The chemiluminescence peak was then recorded by a luminometer.

Nitric oxide (NO) Determination assay: Nitric oxide levels were determined by measuring the accumulated nitrite in the supernatants of cannabidiol derivative-treated peritoneal macrophages, prepared as described hereinabove.

TNF-α determination assay: TNF-α (pg/ml) in cell culture supernatants or in mouse blood plasma was determined by the “sandwich” ELISA technique. ELISA reagents were used according to the manufacturer's protocol (R & D Systems).

### RESULTS

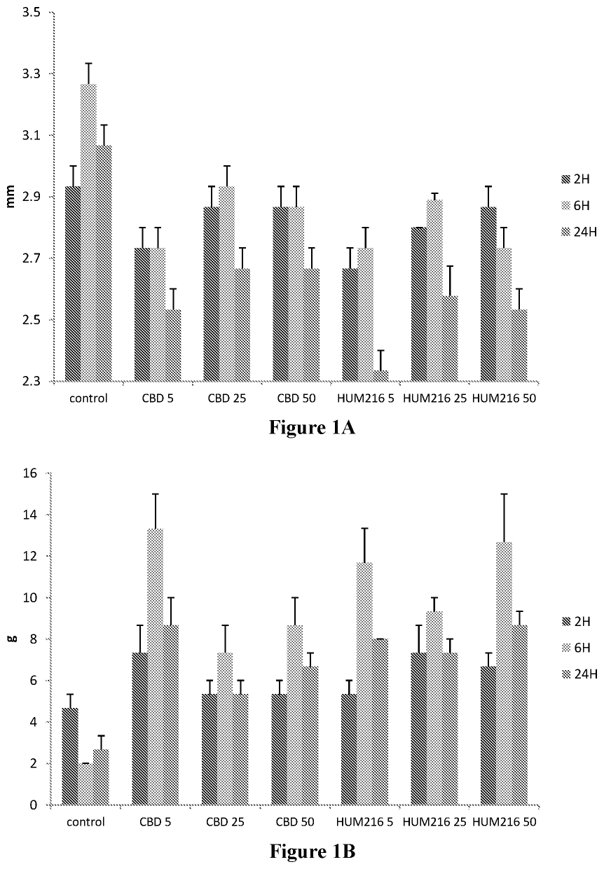

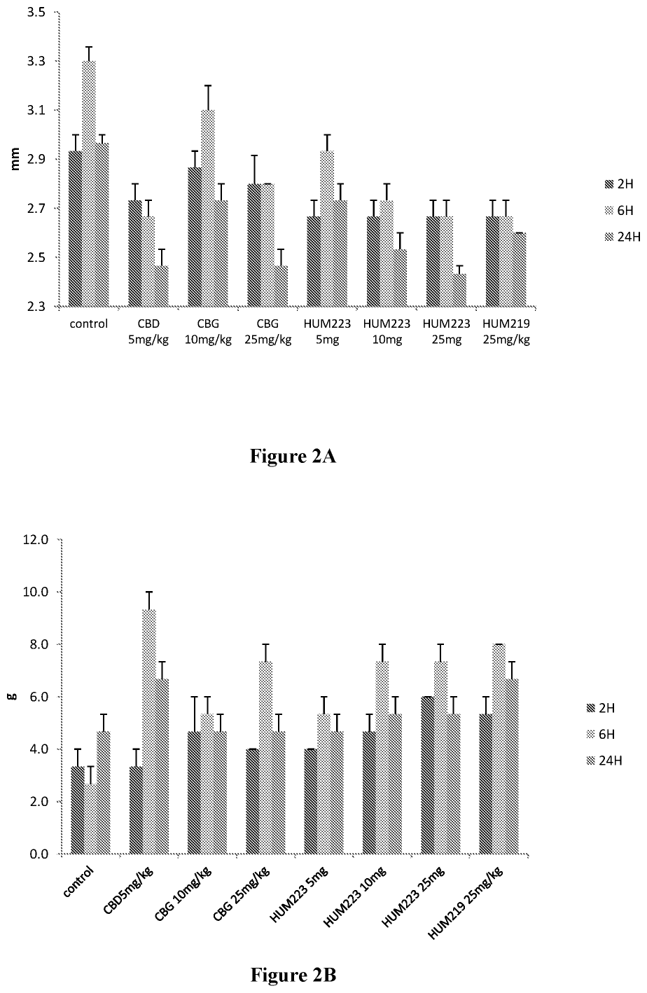

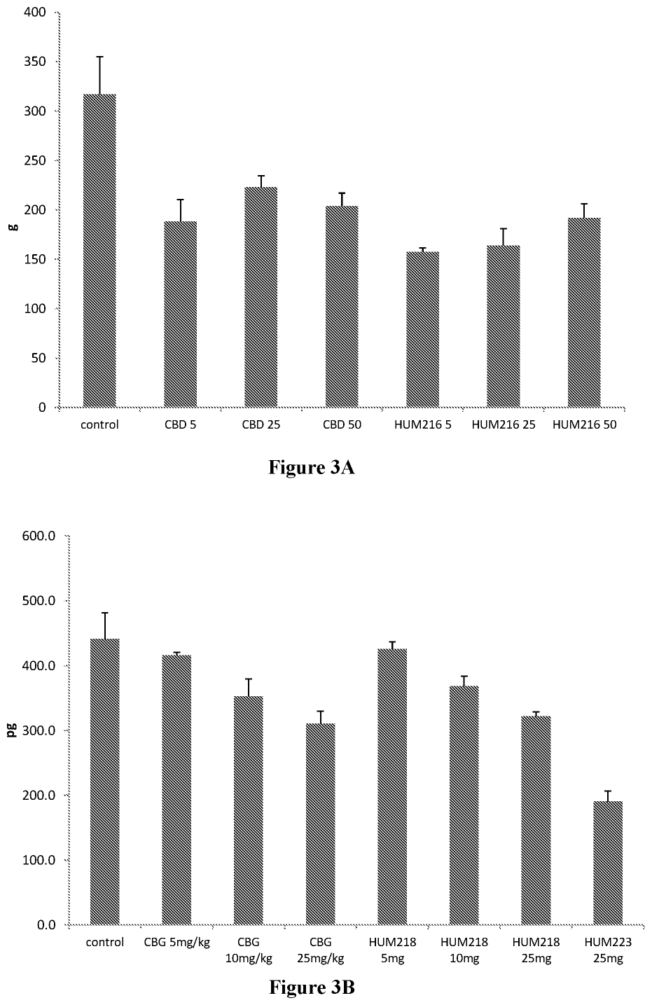

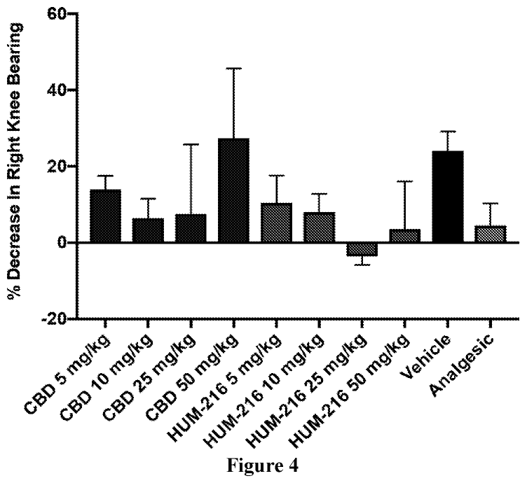

The results of the biological activity assays are presented in FIGS. 1-4.

As can be seen in FIGS. 1-4, the exemplary compounds according to embodiments of the present invention, HUM-216, HUM-217, HUM-218 and HUM-223 all exhibited notable anti-inflammatory activity.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

### REFERENCES

1. Cardillo, B., et al., Gazzetta Chimica Italiana 1973.103(1-2): p. 127-39.
2. Carlini, E. A., et al., Res Commun Chem Pathol Pharmacol, 1975. 12(1): p. 1-15.
3. Weiner, B. Z., et al, European Journal of Medicinal Chemistry, 1975.10(1): p. 79-83.
4. Knaus, E. E., et al., Journal of Chromatographic Science 1976.14(11): p. 525-30.
5. Hendriks, Hv et al., Pharmaceutisch Weekblad, 1978.113(17): p. 413-24.
6. Consroe, P., et al., J Clin Pharmacol, 1981. 21(8-9 Suppl): p. 428S-436S.
7. Jorapur, V. S., et al, Chemistry Letters 1982. 3: p. 299-302.
8. Jorapur, V. S., et al., J Med Chem, 1985. 28(6): p. 783-7.
9. Martin, B. R., et al, NIDA Res. Monogr., 1987. 79: p. 108-22.
10. Mechoulam, Rv et al, NIDA Res Monogr, 1987. 79: p. 15-30.
11. Yamamoto, I., et al., Journal of Pharmacobio-Dynamics 1989.12(8): p. 488-94.
12. Compton, D. R., et al., Journal of Medicinal Chemistry 1990. 33(5): p. 1437-43.
13. Baek, S. H., et al., Bulletin of the Korean Chemical Society, 1992.13(2): p. 117-18.
14. Baek, S. H., et al., Archives of Pharmacal Research, 1992.15(1): p. 5-8.
15. Inoue, S., et al., Nippon Kagaku Kaishi, 1992.1: p. 45-52.
16. Baek, S. H., Bulletin of the Korean Chemical Society, 1993.14(1): p. 144-6.
17. Baek, S. H., et al, Bulletin of the Korean Chemical Society, 1993.14(2): p. 272-4.
18. Tius, M. A., et al., Tetrahedron, 1993. 49(16): p. 3291-304.
19. Baek, S. H., et al., Bulletin of the Korean Chemical Society, 1994.15(6): p. 507-8.
20. Wiley, J. L., et al, J Pharmacol Exp Ther, 2002.301(2): p. 679-89.
21. Sumariwalla, P. F., et al., Arthritis Rheum, 2004. 50(3): p. 985-98.
22. Hanus, L. O., et al., Org Biomol Chem, 2005. 3(6): p. 1116-23.
23. Mechoulam, Rv et al., Phytochemistry Reviews 2005. 4(1): p. 11-18.
24 Padgett, L. W., Life Sci, 2005. 77(14): p. 1767-98.
25. Tchilibon, S., et al., Org Lett, 2000. 2(21): p. 3301-3.
26. R. Mechoulam, Z. Ben-Zvi, J. Chem. Soc. D Chem. Commun. 1969, 343.
27. H. Edery, Y. Grunfeld, G. Porath, Z. Ben-Zvi, A. Shani, R. Mechoulam, Arzneimittelforschung. 1972, 22, 1995-2003.
28. R. L. Yong, X. Wang, Bull. Korean Chem. Soc. 2005, DOI 10.5012/bkcs.2005.26.12.1933.
29. H. Lin, T. Annamalai, P. Bansod, Y.-C. Tse-Dinh, D. Sun, n.d., DOI 10.1039/c3md00238a.
30. M. Černý, J. Málek, Collect. Czechoslov. Chem. Commun. 1970, DOI 10.1135/cccc19702030.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A compound of general formula (I), including any stereoisomer, solvate or salt thereof: 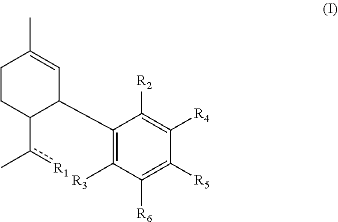
Wherein [symbol]
is a single or double bond;
R1 is selected from CH3 and CH2;
R2 and R3 are selected from OH, O(C1-C3 alkyl), straight or branched C3-C10 alkyl;
R4 is a straight or branched C1-C5 alkyl;
R5 is selected from OH, straight or branched C3-C10 alkyl;
R6 is selected from H, straight or branched C1-C5 alkyl;
Provided that when [symbol] is a double bond at least one of R2 and R3 is different than OH;
or R6 is different than H; and provided that when R3 and R5 are both OH than R2 is C5 alkyl and R4 is C1-C5 alkyl.

2. A compound according to claim 1, wherein [symbol] is a single bond.

3. A compound according to claim 1, wherein [symbol] is a single bond; R2 and R3 are both OH.

4. A compound according to claim 1, wherein R2 and R3 are each O(C1-C3 alkyl).

5. A compound according to claim 1, wherein R2 is a straight or branched C3-C10 alkyl.

6. A compound according to claim 1, wherein R3 and R5 are both OH.

7. A compound according to claim 1, wherein R3 and R5 are both OH; R2 is a straight or branched C3-C10 alkyl; R4 is straight or branched C1-C5 alkyl.

8. A compound according to claim 1, wherein R4 and R6 are each C1-C5 alkyl.

9. A compound according to claim 1, being selected from:
(1′S,2′S)-2′-isopropyl-3,5′-dimethyl-4-pentyl-1′,2′,3′,4′-tetrahydro-[1,1′-biphenyl]-2,6-diol (HUM-217);
(1R,2R)-2′,6′-dimethoxy-3′,5-dimethyl-4′-pentyl-2-(prop-1-en-2-yl)-1,2,3,4-tetrahydro-1,1′-biphenyl (HUM-219);
(1′R,2′R)-5,5′-dimethyl-6-pentyl-2′-(prop-1-en-2-yl)-1′,2′,3′,4′-tetrahydro-[1,1′-biphenyl]-2,4-diol (HUM-229);
(1′R,2′R)-3,5,5′-trimethyl-4-pentyl-2′-(prop-1-en-2-yl)-1′,2′,3′,4′-tetrahydro-[1,1′-biphenyl]-2,6-diol (HUM-236).

10. A compound of general formula (II), including any stereoisomer, solvate or salt thereof: 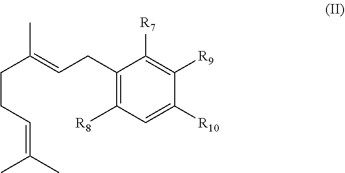
Wherein
R7 and R8 are each selected from OH, O(C1-C3 alkyl), OC(═O)R16;
R9 is selected from H, or C1-C3 alkyl;
R10 is a straight or branched C3-C10 alkyl;
R16 is C1-C5 alkyl optionally substituted by at least one of morpholino, (O(CH2CH2)2NH).
Provided that when both R7 and R8 are OH, then R9 is different than Clalkyl.

11. A compound according to claim 10, wherein at least one of R7 and R8 is O(C1-C3 alkyl).

12. A compound according to claim 10, wherein at least one of R7 and R8 is OC(═O)R16 wherein R16 is C1-C5 alkyl optionally substituted by at least one of morpholino.

13. A compound according to claim 10, wherein both of R7 and R8 are O(C1-C3 alkyl).

14. A compound according to claim 10, being selected from:
((E)-2-(3,7-dimethylocta-2,6-dien-1-yl)-4-methyl-5-pentylbenzene-1,3-diol (HUM-218);
(E)-2-(3,7-dimethylocta-2,6-dien-1-yl)-3-methoxy-5-(2-methyloctan-2-yl)phenol (HUM-223);
(E)-2-(3,7-dimethylocta-2,6-dien-1-yl)-3-methoxy-5-pentylphenyl 3-morpholinopropanoate (HUM-233);
(E)-4-(3-(2-(3,7-dimethylocta-2,6-dien-1-yl)-3-methoxy-5-pentylphenoxy)-3-oxopropyl)morpholin-4-ium (Z)-3-carboxyacrylate (HUM-234);
(E)-2-(3,7-dimethylocta-2,6-dien-1-yl)-1,3-dimethoxy-4-methyl-5-pentylbenzene (HUM-235).

15. (canceled)

16. (canceled)

17. (canceled)

18. (canceled)

19. (canceled)

20. (canceled)

21. (canceled)

22. (canceled)

23. (canceled)

24. (canceled)

25. A method of treating a condition, disease or disorder associated with at least one of inflammation, obesity, and pain; said method comprising administering to a subject in need thereof a therapeutically effective amount of at least one compound of claims 1 or 10.

26. (canceled)

27. The method according to claim 25, wherein said medical condition, disease or disorder is selected from the group consisting of rheumatoid arthritis, atherosclerosis, Crohn's disease and multiple sclerosis.

28. The method of claim 25, wherein said treatment is a non-abusive treatment.

29. (canceled)

30. (canceled)

31. (canceled)

32. (canceled)
